(19) United States
(12) Plant Patent Application Publication
(10) Pub. No.: US 2007/0107106 P1
(43) Pub. Date: May 10, 2007

(54) ABUTILON PLANT NAMED 'ATN YB5'

(76) Inventor: Lucilene Anatriello, Sao Jose do Rio Preto (BR)

Correspondence Address: ALBERT O COTA, 5460 WHITE OAK AVE, SUITE A-331, ENCINO, CA, 91316, US

(21) Appl. No.: 11/266,037

(22) Filed: Nov. 4, 2005

(51) Int. Cl. A01H 5/00

(52) U.S. Cl. PLT/226

(57) ABSTRACT

A new and distinct Abutilon plant cultivar is disclosed, characterized by bright yellow flowers, large, glossy leaves, an upright, vigorous growth habit and a tall plant height. The new variety is an Abutilon, and naturally blooms from April to September.

### LATIN NAME OF THE GENUS AND SPECIES

Abutilon vexillarium

### VARIETY DENOMINATION

‘ATN YB5’

### BACKGROUND OF THE INVENTION

The new cultivar is a product of a planned breeding program. The new variety was discovered as a naturally occurring whole plant mutation. It was discovered by Lucilene Anatriello in February 2004 in São José do Rio Preto, Brazil.

Asexual reproduction of the new cultivar ‘ATN YB5’ by vegetative cuttings was performed in São José do Rio Preto, Brazil and has shown that the unique features of this cultivar are stable and reproduced true to type on successive generations.

### SUMMARY OF THE INVENTION

The cultivar ‘ATN YB5’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of‘ATN YB5.’ These characteristics in combination distinguish ‘ATN YB5’ as a new and distinct Abutilon cultivar:
1. Tall, vigorous growth habit;
3. Large green leaves;
5. Long blooming period; and
6. Bright yellow petals.

Plants of the new cultivar ‘ATN YB5’ are similar to plants of the parent, ‘Abutilon Yellow’, in most horticultural characteristics, however, plants of the new cultivar ‘ATN YB5’ are taller and more vigorous than the parent. Additionally, the new variety has larger leaves as well as more leaves per lateral branch. The new variety resists dropping leaves and blooms one month longer than the parent variety. The new variety ‘ATN YB5’ also has brighter yellow petals.

There is no similar commercial varieties available to compare to ‘ATN YB5’.

### BRIEF DESCRIPTION OF THE PHOTOGRAPH

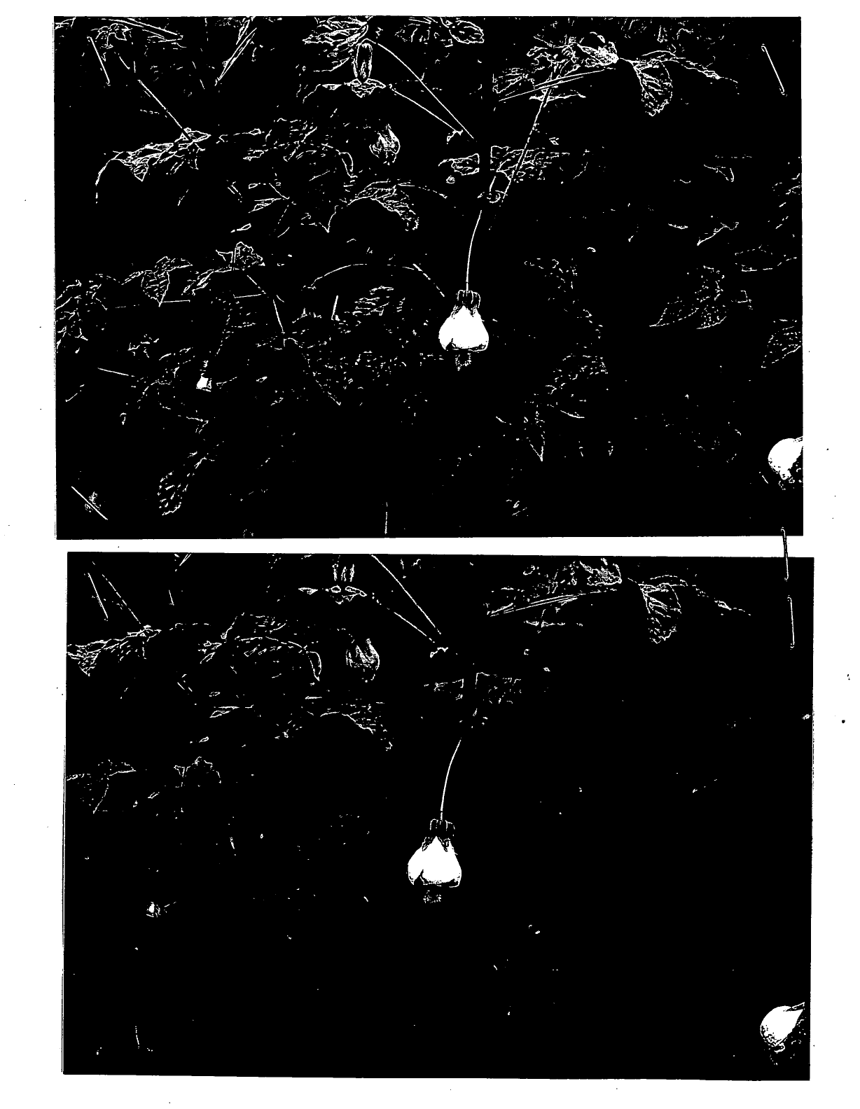

The accompanying photograph in FIG. 1 illustrates in full color a typical plant of ‘ATN YB5’ grown in an outdoor garden with a grouping of 15 other plants. The colors are as nearly true as is reasonably possible in a color representative of this type.

### DETAILED BOTANICAL DESCRIPTION

In the following description, color references are made to The Royal Horticultural Society Colour Chart except where general terms of ordinary dictionary significance are used. The following observations and measurements describe ‘ATN YB5’ plants grown outdoors in Oxnard, Calif. from November 2004 to August 2005. The growing temperature ranged from 10° C. to 15° C. at night to 12° C. to 30° C. during the day. Measurements and numerical values represent averages of typical flowering types.
Botanical classification: Abutilon vexillarium cultivar ‘ATN YB5.’
Propagation:
Time to rooting: 15 to 21 days at approximately 21° C.
Root description: Fine, fibrous.
Plant:
Growth habit: Upright shrub.
Height: Approximately 170.18 cm.
Blooming period: April to September.
Plant spread: Approximately 73.66 cm.
Growth rate: Highly vigorous.
Branching characteristics: Moderate upright branching.
Length of lateral branches: Approximately 150 cm.
Number of leaves per lateral branch: Approximately 46.
Age of plant described: Approximately 221 days.
Foliage:
Leaf:
   Arrangement.—Alternate.
   Average length.—Approximately 9.2 cm.
   Average width.—Approximately 13.5 cm.
   Shape of blade.—Palmate.
   Apex.—Acuminate
   Base.—Hastate.
   Attachment.—Stalked.
   Margin.—Entire.
   Texture of top surface.—Lightly pubescent.
   Texture of bottom surface.—Smooth.
   Leaf internode length.—Approximately 5 cm.
   Color.—Young foliage upper side: Near R.H.S. Green 137A. Young foliage underside: Near Green 137C. Mature foliage upper side: Near Green 137A. Mature foliage under side: Near Green 137C.
   Venation.—Type: Palmately net. Venation color upper side: Near Yellow-Green 144C. Venation color under side: Near Yellow-Green 144C.
Petiole:
   Average length.—Approximately 6.6 cm.
   Color.—Near Yellow-Green 144B.
   Diameter.—Approximately 0.01 cm.
Flower:
Bud:
   Bud shape.—Spherical.
   Bud length.—Approximately 2 cm.
   Bud diameter.—Approximately 1.2 cm.
   Bud color.—Near Near Yellow 12A.
   Shape.—Bell-shape, with five unfused petals.
Flower:
   Facing direction.—Down.
   Type.—Simple.
   Aspect.—Concave.
   Diameter of entire flower.—Approximately 3.5 cm.
   Length of flower.—Approximately 3.9 cm.
Petals:
   Length of petal.—Approximately 3.5 cm.
   Width of petal.—Approximately 3 cm.
   Apex shape of petal.—Obtuse.
   Petal margin.—Entire.
Color:
   Upper surface at first opening.—Near Yellow 12B.
   Upper surface at maturity.—Near Yellow 12A.
   Upper surface at fading.—Near Yellow 13C.
   Under surface at first opening.—Near Yellow 11A.
   Under surface at maturity.—Near Yellow 12A.
   Under surface at fading.—Near Yellow 13C.
Fragrance: None.
Sepal:
Number: 5; fused at base.
Sepal length: Approximately 2.2 cm.
Sepal width: Approximately 0.06 cm.
Apex shape: Acute.
Margin: Entire.
Color: Inside sepal color near R.H.S. Yellow-Green 145B and outside sepal color near R.H.S. Yellow-Green 145A.
Reproductive organs: 1 normal pistil and over 100 normal stamens.
Peduncle:
   Width.—Approximately 0.01 cm.
   Length.—Approximately 14.6 cm in length.
   Angle to stem.—Downward from stem at 135° angle.
   Strength.—Strong.
   Texture.—Smooth.
   Color.—Near Yellow-Green 143B.
Reproductive organs:
   Number of pistils per floret.—1.
   Stigma shape.—Globular.
   Stigma color.—Near Yellow-Orange 14B.
   Style color.—Near Yellow 8A.
   Style length.—Approximately 3.4 cm.
   Stamens.—Over 100 normal stamens.
   Anther shape.—Globular.
   Anther color.—Near Yellow 14A.
   Pollen color.—Near Yellow 14A.
Other characteristics:
Disease resistance: Neither resistance nor susceptibility to diseases or pests has-been observed in this variety.
Drought tolerance and cold tolerance: Hardy to 23° F. (−5° C.) and will generally grow back from the base if the top is frozen. No drought tolerance has been observed.
Fruit/seed production: Seed production has not been observed.

What is claimed is:

1. A new and distinct Abutilon plant cultivar named ‘ATN YB5’ characterized by red and yellow flowers, large gloss leaves, an upright, vigorous growth habit and a tall plant height.
